(19) United States
(12) Patent Application Publication
(10) Pub. No.: US 2004/0241110 A1
(43) Pub. Date: Dec. 2, 2004

(54) Jelly-type tooth-bleaching patch

(76) Inventor: Sang-Ho Lee, Sungnam (KR)

Correspondence Address: Galgano & Burke, LLP, Suite 135, 300 Rabro Drive, Hauppauge, NY, 11788, US

(21) Appl. No.: 10/854,542

(22) Filed: May 26, 2004

(30) Foreign Application Priority Data
May 30, 2003 (KR) 10-2003-0034927

(51) Int. Cl. A61K 7/16; A61K 7/20

(52) U.S. Cl. 424/53; 433/217.1

(57) ABSTRACT

The present invention relates to a tooth-bleaching patch, and more particularly, to a jelly-type tooth-bleaching patch which comprises a jelly-type adhesive containing a substance showing a tooth-bleaching effect. By this jelly-type adhesive, the tooth-bleaching patch is convenient to use, and it is flexibly attached to teeth in accordance with the teeth shape and prevents a tooth-bleaching agent from being excessively exposed to oral saliva, so that the tooth-bleaching agent can come in contact with the teeth at a sufficient concentration for a sufficient time to bleach the teeth, so as to maximize its tooth-bleaching effect.

### BACKGROUND OF THE INVENTION

1. Field of the Invention

The present invention relates to a jelly-type tooth-bleaching patch showing a tooth bleaching effect upon attachment to teeth.

2. Description of the Prior Art

Materials for tooth bleaching include peroxides and agents for the removal and prevention of tartar. Tooth bleaching using peroxides has been mainly performed in dental clinics, since peroxides have a harmful effect on the skin when they come in contact with the skin. However, this tooth bleaching using peroxides is troublesome and significantly expensive. Thus, an interest on the development of products capable of achieving a tooth bleaching effect in a simple manner is being concentrated.

U.S. Pat. No. 5,891,453 discloses a gel-type tooth-whitening strip. However, this tooth-whitening strip has a drawback in that an adhesive contained in the tooth-whitening strip are sticky to the hand or gum. In an attempt to solve this problem, a dry-type patch can be used. However, this dry-type patch can be attached only to the front of teeth due to its insufficient flexibility, and cannot contain a sufficient amount of hydrogen peroxide as a main material to bleach teeth, because of a limitation that the hydrogen peroxide must be contained in its thin solid coating film. For this reason, it has a reduced teeth-bleaching effect. Furthermore, the dry-type patch contains an excessive amount of a stabilizer for its stabilization in heating and drying steps and for the prevention of crystallization of inorganic salts. In addition, the dry-type patch releases hydrogen peroxide within 10 minutes after its attachment to teeth at such a fast rate that it irritates the gum, and the released hydrogen peroxide is diluted in saliva, so that hydrogen peroxide in the dry-type patch does not remain at a sufficient concentration to bleach teeth.

### SUMMARY OF THE INVENTION

Accordingly, the present invention has been made to solve the above-mentioned problems occurring in the prior art, and an object of the present invention is to provide a jelly-type tooth-bleaching patch which has an adhesiveness similar to a poultice for attachment to the skin, so that the patch is convenient to use (e.g., it is not stuck to hand).

Another object of the present invention is to provide a jelly-type tooth-bleaching patch which comprises an adhesive that absorbs saliva at a very slow rate without releasing peroxide, so that the time taken for peroxide to dissolve in saliva is lengthened to increase the contact time of peroxide with teeth, such that a sufficient concentration of peroxide to bleach the teeth comes in contact with the teeth, thereby maximizing its tooth bleaching effect.

Still another object of the present invention is to provide a jelly-type tooth-bleaching patch which removes bad breath and sterilizes harmful microorganisms in the mouth, thereby preventing gun diseases.

To achieve the above objects, the present invention provides a jelly-type tooth-bleaching patch comprising: a film, an adhesive applied on the film, and a moisture-proof wrapping paper adhered on the adhesive.

### BRIEF DESCRIPTION OF THE DRAWINGS

FIG. 1 shows the structure of a jelly-type tooth-bleaching patch according to the present invention; and

FIG. 2 is a plan view of a jelly-type tooth-bleaching patch according to the present invention.

### DETAILED DESCRIPTION OF THE PREFERRED EMBODIMENTS

In a preferred embodiment, the adhesive in the jelly-type tooth-bleaching patch according to the present invention comprises peroxide, a peroxide stabilizer, an inhibitor of tartar formation, a coating-forming agent, a coating-forming aid, a wetting agent, and a plasticizer.

In a preferred embodiment, the adhesive in the jelly-type tooth-bleaching patch comprises a coating-forming peroxide, a peroxide stabilizer, an inhibitor of tartar formation, a coating-forming agent, a coating-forming aid, a wetting agent, and a plasticizer.

In a preferred embodiment, the peroxide in the adhesive is at least one compound selected from the group consisting of hydrogen peroxide, carbamide peroxide, calcium peroxide, sodium percarbonate, sodium perborate, tetra sodium pyrophosphate peroxide, hydrogen peroxide 2-pyrolidinone 1-ethenyl-homopolymer, PVP-VA-hydrogen Peroxide®, Gafquat HS-100-hydrogen peroxide®, PVP-AA-hydrogen peroxide®, and Ganex-hydrogen peroxide®.

In a preferred embodiment, the peroxide stabilizer in the adhesive is at least one compound selected from the group consisting of sodium stannate, alkylarylsulfonate, alkylarylsulfonate salt, alkyldiphenyloxide disulfonate, Span 20®, Span 40®, Span 60®, Span 85®, Tween®, and polyoxyethylene glycol hydroxymethylcellulose.

In a preferred embodiment, the inhibitor of tartar formation in the adhesive is sodium salt, calcium salt, or a mixture thereof

In a preferred embodiment, the sodium salt which is used as the inhibitor of tartar formation is at least one compound selected from the group consisting of tetrasodium pyrophosphate (TSPP), sodium hexametaphosphate (SHMP), sodium acid pyrophosphate (SAPP), sodium pyrophosphate (TKPP), acidic sodium meta-polyphosphate and acidic sodium polyphosphate.

In a preferred embodiment, the calcium salt as the inhibitor of tartar formation is at least one compound selected from the group consisting of potassium benzoate, potassium citrate, potassium fluoride, potassium bicarbonate, potassium carbonate, potassium chloride, potassium lactate, potassium pyrophosphate, and potassium phosphate.

In a preferred embodiment, the coating-forming agent in the adhesive is at least one compound selected from the group consisting of polyvinyl pyrrolidone, polyvinyl alcohol, sodium alginate, hydrogen peroxide 2-pyrolidinone 1-ethenyl-homopolymer, Gafquat HS-100-hydrogen peroxide®, PVP-AA-hydrogen peroxide®, and Ganex-hydrogen peroxide®.

In a preferred embodiment, the coating-forming aid in the adhesive is at least one compound selected from the group consisting of methyl cellulose, dextrin, urea, hydroxy propyl starch, gelled starch, carboxymethyl cellulose, carboxypropyl cellulose, poloxamer, carrageenan, vee gum, karaya gum, xanthan gum, guar gum, hydroxypropylmethyl cellulose, gum arabic, gum fra, hydroxyethyl cellulose, hydroxypropyl cellulose, hydroxymethyl cellulose, phthalate acetate cellulose, ethyl cellulose, phthalate hydroxypropylmethyl cellulose, calcium carboxymethyl cellulose, and sodium carboxymethyl cellulose.

In a preferred embodiment, the wetting agent in the adhesive is at least one compound selected from the group consisting of polyethylene glycol, glycerin, polypropylene glycol, polybutylene glycol (1,3-butandiol), bentnite, and sorbitol.

In a preferred embodiment, the plasticizer in the adhesive is at least one compound selected from the group consisting of caster oil, hydrogenated caster oil, and silicone oil.

In a preferred embodiment, the coating-forming peroxide in the adhesive is at least one compound selected from the group consisting of hydrogen peroxide 2-pyrolidinone 1-ethenyl-homopolymer, PVP-VA-hydrogen peroxide®, Gafquat HS-100-hydrogen peroxide®, PVP-AA-hydrogen peroxide®, and Ganex-hydrogen peroxide®.

In a preferred embodiment, the adhesive additionally comprises a sweetening agent.

In a preferred embodiment, the adhesive comprises 0.5-10 wt % of the peroxide, 0.5-5 wt % of the peroxide stabilizer, 0.1-1.5 wt % of the inhibitor of tartar formation, 6.1-15 wt % of the coating-forming agent, 1-10 wt % of the coating-forming aid, 10-70 wt % of the wetting agent, 0.1-3 wt % of the plasticizer, and 0.01-1 wt % of the sweetener.

In a preferred embodiment, the adhesive comprises hydrogen peroxide at the amount of 0.5-3 wt % relative to the total weight of the adhesive.

In a preferred embodiment, the adhesive comprises hydrogen peroxide at the amount of 3-10 wt % relative to the total weight of the adhesive.

In a preferred embodiment, the adhesive comprises a crosslinking agent and a crosslinking aid.

In a preferred embodiment, the coating-forming agent contains at least one compound selected from the group consisting of polyacrylic acid, sodium polyacrylate, polyacrylic acid/sodium polyacrylate copolymer, PVP/VA copolymer, VP/acrylates/lauryl methacrylate copolymer, polymethyl vinyl ether/maleic anhydride, vinyl acetate/vinyl neodecanoate/maleic half-ester, poly N-vinyl formamide, vinyl acetate/crotonic acid/vinyl neodecanoate copoylmer, polyvinyl acetate, polymethyl methacrylate, methacrylic acid copolymer, aminoalkyl methacrylate copolymer, cellulose acetate phthalate, shellac, vinyl acetate/crotonic acid/vinyl propionate copolymer, and methaacryloyl ethyl betaine/methacrylate copolymer.

In a preferred embodiment, the crosslinking agent is at least one compound selected from the group consisting of salicylic acid, gluconic acid, silicic acid, malic acid, tartaric acid, glycolic acid, and lactic acid.

In a preferred embodiment, the crosslinking aid is at least one compound selected from the group consisting of kaoline, hydroxy aluminum aminoacetate, dried aluminum hydroxide powder, aluminum silicate, aluminum-magnesium hydroxide, synthetic hydrotalcite, cum lite®, and aluminum hydroxide gel.

In a preferred embodiment, the adhesive comprises 0.1-10 wt % of the peroxide, 0.5-5 wt % of the peroxide stabilizer, 0.1-5 wt % of the inhibitor of tartar formation, 0.1-10 wt % of the coating-forming agent, 0.1-10 wt % of the coating-forming aid, 10-70 wt % of the wetting agent, 0.1-3 wt % of the plasticizer, 0.1-2 wt % of the sweetener, 0.1-1 wt % of the crosslinking agent, and 0.01-0.6 wt % of the crisslinking aid.

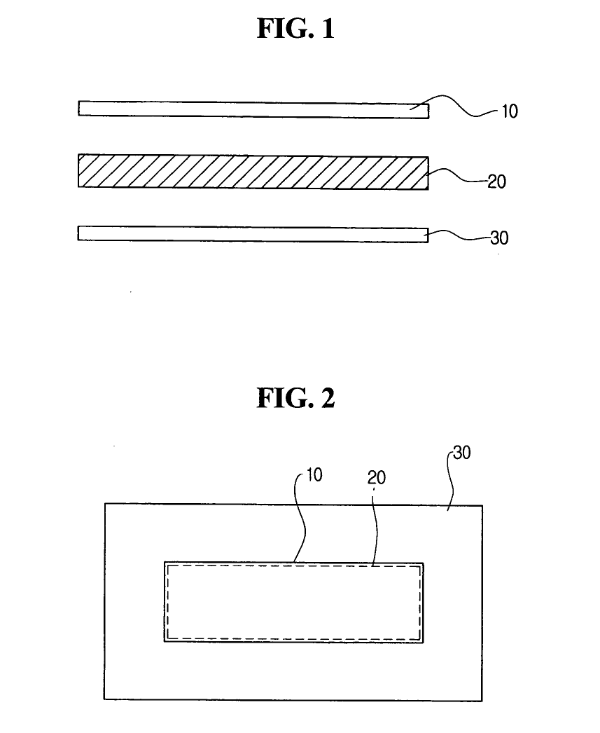

As shown in FIG. 1, the jelly-type tooth-bleaching patch according to the present invention comprises a flexible film 30, an adhesive 20, and a moisture proof wrapping paper 10. As the flexible film, a PET film is used, and as the moisture-proof wrapping paper, a polypropylene film is used.

For the effects of the effects of tooth bleaching, tartar formation prevention and sterilization, the adhesive in the jelly-type tooth-bleaching patch according to the present invention contains peroxide and the inhibitor of tartar formation. Also, in order to maintain the adhesive in a jelly form, the adhesive contains the coating-forming agent, the coating-forming aid, the plasticizer, the cosslinking agent and the crosslinking aid. Particularly, the coating-forming aid, the crosslinking agent and the crosslinking aid are critical to maintain the adhesive in a jelly form.

The peroxide contained in the adhesive has the effects of tooth bleaching, bad breath removal, and sterilization and thus gum disease prevention. As the peroxide, hydrogen peroxide itself or hydrogen peroxide salt is used. Examples of the hydrogen peroxide salt include carbamide peroxide, calcium peroxide, sodium percarbonate, sodium perborate and tetrasodium pyrophosphate peroxide. Examples of current commercial peroxide include peroxydone K-30® (ISP Co.), Gafquat HS-100-hydrogen peroxide®, PVP-AA-hydrogen peroxide®, and Ganex-hydrogen peroxide®. Of them, peroxydone K-30® (ISP Co.) is hydrogen peroxide 2-pyrolidinone 1-ethenyl homopolymer. In the present invention, the hydrogen peroxide, the hydrogen peroxide salt, the peroxide sold under the above-mentioned trademarks, and a mixture thereof, is used as the peroxide. The content of hydrogen peroxide in the peroxide product sold under the above-mentioned trademarks is 17-30 wt % for peroxydone K-30® (ISP Co.), 20 wt % for Gafquat HS-100-hydrogen peroxide®, 10 wt % for PVP-VA-hydrogen peroxide®, and 10 wt % for Ganex-hydrogen peroxide®. The content of the hydrogen peroxide in the adhesive is important, because when the amount of the hydrogen peroxide contained in the peroxide is more than 3 wt % based on the total weight of the adhesive, this adhesive is classified as a medical product, so that the other components in the adhesive, particularly the coating-forming agent, must be made of a material harmless to the human body. On the other hand, when the content of the hydrogen peroxide in the adhesive is less than 3 wt %, this adhesive is used in the preparation of non-medical products, such as cosmetics. Accordingly, in the preparation of the medical adhesive, the kind of the coating-forming agents as described below and the content of hydrogen peroxide in each of peroxide need to be considered.

As the inhibitor of tartar formation, polyphosphate or calcium salt is used. This tartar formation inhibitor enhances the tooth-bleaching effect of the peroxide.

The coating-forming peroxide functions as both the coating-forming agent and the peroxide, and may be automatically formed into a certain degree of a coating film in the absence of the coating-forming agent. Examples of this substance include the commercial peroxide products as described above, namely, hydrogen peroxide 2-pyrolidinone 1-ethenyl-homopolymer, PVP-VA-hydrogen peroxide®, Gafquat HS-100-hydrogen peroxide®, PVP-AA-hydrogen peroxide®, and Ganex-hydrogen peroxide®. The coating-forming peroxide can be used alone without a distinction between the peroxide and the coating-forming agent. In this case, however, the peroxide and the coating-forming agent may additionally be used.

The coating-forming aid is a substance that helps the formation of a coating film by forming a frame on which the coating film can be formed. Thus, the coating film is easily formed and the formed coating film has high strength so that a jelly-type adhesive can be formed. As the coating-forming aid, cellulose materials and preferably water-soluble celluloses are preferably used, but other polysaccharides or adhesive rubbers may also be used. The amount of the coating-forming aid is the key to the formation of a jelly-type adhesive, and is determined depending on the kind and amount of the components in the adhesive. The coating-forming aid is contained in the adhesive at the amount of 1-10 wt %. The coating-forming aid acts to maintain the peroxide within the adhesive, thus preventing the release of the peroxide.

Polypyrrolidone which is used as the coating-forming agent in the adhesive of the jelly-type tooth-bleaching patch according to the present invention has a molecular weight of 400,000-1,200,000.

If the coating-forming agent contains acrylic components, the crosslinking agent and the crosslinking aid will contribute to the formation of a jelly-type adhesive. An adhesive formed with the coating-forming agent containing acrylic components is a non-medical product, and distinguished from medical products. As the crosslinking agent, an organic acid is used, and as the crosslinking aid, aluminum (Al) or silicon (Si) ions are used. The crosslinking agent and the crosslinking aid are contained at very small amounts of 0.1-1 wt % and 0.01-0.6 wt %, respectively, but when the coating-forming agent containing acrylic components is used, the addition of the crosslinking agent and the crosslinking aid is necessary for the formation of a coating film and the maintenance of the formed coating film in a jelly form. Examples of a material, which is used as the crosslinking aid, include hydroxyl aluminum aminoacetate sold under the trademark glycinal®, and synthetic hydrotalcite sold under the trademark alkamac®. If the crosslinking agent and the crosslinking aid are contained in the adhesive, the coating-forming aid can be contained at a smaller amount than that in the case where the adhesive does not contain the crosslinking agent and the crosslinking aid. In this case, the content of the coating-forming aid in the adhesive is in a range of 0.1-10 wt %.

The wetting agent is a-substance serving to supply moisture to the adhesive. As the wetting agent, glycols (i.e., trivalent alcohols) are used. The wetting agent is used at a sufficient amount to prevent the adhesive from being dried into a dry form, and its amount is preferably 10-70 wt % relative to the total weight of the adhesive. However, the amount of the wetting agent is determined depending on the kind and amount of the other components in the adhesive. If an excessive amount of the wetting agent is used, the adhesive will become a gel form, and if it is used at an insufficient amount, the adhesive will be dried.

The plasticizer is oils and is not contained at large amounts like that of the wetting agent. It is used at a small amount of 0.1-3 wt %, and serves to increase the flexibility of the adhesive and to improve the feeling and adhesion to teeth of the tooth-bleaching patch.

The present invention will hereinafter be described in further detail by examples. It will however be obvious to a person skilled in the art that the present invention is not limited to or by the examples.

### EXAMPLES

An adhesive for a jelly-type tooth-bleaching patch was prepared using the components and contents given in Table 1. Then, the adhesive was applied on a PET film as in FIG. 2, and covered with a polyethylene film, thereby preparing a jelly-type tooth-bleaching patch.

TABLE 1

| Component | Example 1 (wt %) | Example 2 (wt %) |
|---|---|---|
| Solvent | Purified water (54.5), ethyl | Purified water (41), ethyl |
| | alcohol (4) | alcohol (5.3) |
| Peroxide | | Hydrogen peroxide (2.6) |
| Peroxide stabilizer | | Sodium stannate (1) |
| Inhibitor of tartar formation | Sodium methaphosphate (2) | Calcium citrate (0.5) |
| Coating-forming agent | Polyvinylpyrrolidone (6.5) | Polyvinyl alcohol (3), |
| | | methacryloyl ethyl |
| | | betaine/methacrylate |
| | | copolymer (0.5) |
| Coating-forming aid | Hydroxypropylcellulose (0.9) | Carboxymethylcellulose |
| | | (2) |
| Coating-forming peroxide | Peroxydone K-30 ® (9) | |
| Wetting agent | Butylene glycol (2), glycerin | Polyoxyethylene glycol |
| | (13.8) | (2), glycerin (33), sorbitol |
| | | (1) |
| Plasticizer | Hydrogenated castor oil (1) | Castor oil (0.5) |
| Sweetening agent | Xyiltol (0.7) | Saccharine sodium (0.1), |
| | | Spearmint oil (0.2) |
| Crosslinking agent | | |
| Crosslinking aid | | |
| Total | 100 | 100 |
| Component | Example 3 (wt %) | Example 4 (wt %) |
| Solvent | Purified water (66) | Purified water (60.3), ethyl |
| | | alcohol (4.8) |
| Peroxide | Hydrogen peroxide (2.6) | Hydrogen peroxide (2.5) |
| Peroxide stabilizer | | |
| Inhibitor of tartar formation | Calcium benzoate (0.5) | Calcium carbonate (0.5) |
| Coating-forming agent | Polyvinylpyrrolidone (0.5), | Polyvinylpyrrolidone (10), |
| | PVA/VA copolymer (4) | polyvinyl acetate (1) |
| Coating-forming aid | Sodium methylcellulose (10), | Calcium carboxymethyl |
| | hydroxypropyl cellulose (0.9) | cellulose (2), |
| | | hydroxypropyl cellulose |
| | | (2) |
| Coating-forming peroxide | | |
| Wetting agent | Glycerin (7.5) | Glycerin (10) |
| Plasticizer | Silicone oil (1), castor oil | Hydrogenated castor oil |
| | (0.5) | (1), silicone oil (1) |
| Sweetening agent | Spearmint oil (0.2) | Saccharine calcium (0.2), |
| | | Spearmint oil (0.2) |
| Crosslinking agent | | |
| Crosslinking aid | | |
| Total | 100 | 100 |
| Component | Example 1 (wt %) | Example 2 (wt %) |
| Solvent | Purified water (6), ethyl | Purified water (42.7) |
| | alcohol (4.5) | |
| Peroxide | | Urea hydrogen peroxide |
| | | (8) |
| Peroxide stabilizer | Alkyldiphenyl oxide | Span 20 ® (3) |
| | disulfonate (1.5) | |
| Inhibitor of tartar | Acidic sodium pyrophosphate | Acidic sodium |
| formation | (0.5) | pyrophosphate (5) |
| Coating-forming agent | Polyacrylic acid (3.3) | Polyacrylic acid/sodium |
| | | polyacrylate copolymer |
| | | (7), cellulose acetate (4) |
| Coating-forming aid | Calcium | |
| | carboxymethylcellulose (2), | |
| | hydroxypropylcellulose (0.9) | |
| Coating-forming peroxide | Peroxydone K-30 ® (10) | |
| Wetting agent | Glycerin (70) | Glycerin (29) |
| Plasticizer | Hydrogenated castor oil (1) | |
| Sweetening agent | Spearmint oil (0.3) | Spearmint (0.2) |
| Crosslinking agent | | Tartaric acid (1) |
| Crosslinking aid | | Glycinal ® (0.1) |
| Total | 100 | 100 |
| Solvent | Purified water (70) | Purified water (65.2) |
| Peroxide | Hydrogen peroxide (2.9) | Sodium percarbonate (2.4) |
| Peroxide stabilizer | | Alkylaryl sulfonate (1) |
| Inhibitor of tartar | Sodium | Acidic sodium |
| formation | hexamethaphosphate (2) | polyphosphate (5) |
| Coating-forming agent | Polyacrylic acid (2), | Polyacrylic acid/sodium |
| | polyacrylic acid/sodium | polyacrylate copolymer (6.2) |
| | polyacrylate copolymer (5) | |
| Coating-forming aid | Sodium | Sodium |
| | Hydroxypropylenecellulose | carboxymethylcellulose (0.9) |
| | (0.9) | |
| Coating-forming peroxide | | |
| Wetting agent | Glycerin (14), polyethylene | Glycerin (13.3), |
| | glycol (2) | polyoxyethylene glycol (2), |
| | | propylene glycol (2) |
| Plasticizer | Silicone oil (1) | |
| Sweetening agent | Saccharine (0.2) | Papain (2) |
| Crosslinking agent | Salicylic acid (0.2) | Tartaric acid (0.5) |
| Crosslinking aid | Dried aluminum hydroxide | Kaolin (0.5), hydrotalcite (1) |
| | (0.4) | |
| Total | 100 | 100 |

### Experimental Example

The jelly-type tooth-bleaching patch having the composition of Example 1 was attached to teeth, and then, subjected to the following experiment in order to determine mouth irritation by the amount of a tooth-bleaching component (i.e., hydrogen peroxide) released from the adhesive into oral saliva, and to determine the tooth-bleaching effect of the inventive jelly-type tooth-bleaching patch by the contact time and amount of hydrogen peroxide with teeth.

As a control group, the commercially available dry-type bleaching patch “Claren®” (sold from LG Household & Health Care Ltd., Korea) was used.

1. Measurement of Content of Hydrogen Peroxide in Patch

In order to measure the total content of hydrogen peroxide in each of the jelly-type and dry-type bleaching patches, 800 g of distilled water was used as a solvent. To maximize the dissolution of hydrogen peroxide contained in the patches, the distilled water was used at 40° C. and the patches were stirred. The dissolution of the patches was conducted for 10 minutes. Thereafter, analytical test strips (Merck Co.) were used to measure the concentration of hydrogen peroxide in the distilled water. The measurement results are given in Table 2 below.

TABLE 2

| | Jelly-type | Dry-type |
|---|---|---|
| | tooth-bleaching | tooth-bleaching |
| | patch | patch |
| One sheet of tooth- | 5.3 ppm | 5 ppm |
| bleaching patch + 800 g of | | |
| distilled water | | |
| Amount of hydrogen | 4.24 ppm | 4.00 ppm |
| peroxide | | |

The results in Table 2 above shows that the amounts of hydrogen peroxide contained in the patches are substantially similar to each other.

2. Amount of Hydrogen Peroxide Released

To measure the amount of hydrogen peroxide released into oral saliva, the patches were attached to teeth, and saliva spat after every 10 minutes and was diluted in 500 g of distilled water while the concentration of hydrogen peroxide was measured. The measurement results are given in Table 3 below.

TABLE 3

| | Jelly-type | | Dry-type | |
|---|---|---|---|---|
| | tooth-bleaching | | tooth-bleaching | |
| Lapsed time | patch | | patch | |
| 10 min | 0.1-0.2 | ppm | 1 | ppm |
| 20 min | 0.1-0.2 | ppm | 1 | ppm |
| 30 min | 0.1-0.2 | ppm | 1 | ppm |
| 40 min | 0.1-0.2 | ppm | 1 | ppm |
| 50 min | 0.1 | ppm | 1 | ppm |
| 60 min | 0.1 | ppm | 0.9 | ppm |

The measurement results in Table 3 above shows that the amount of hydrogen peroxide released from the jelly-type tooth-bleaching patch into oral saliva after the lapse of each time interval is only 10% of the dry-type tooth-bleaching patch. This suggests that the content of hydrogen peroxide in the patches is larger in the jelly-type tooth-bleaching patch than that in the dry-type tooth-bleaching patch, and thus, the hydrogen peroxide in the dry-type patch remains in the patch in contact with teeth for a longer time than that of the dry-type patch.

The amount of hydrogen peroxide released into saliva for one hour was calculated from the results of Table 3, and the calculated results are given in Table 4 below. Furthermore, the percent reduction of hydrogen peroxide in the patch after one hour was calculated.

TABLE 4

| | Jelly-type | Dry-type |
|---|---|---|
| | tooth-bleaching | tooth-bleaching |
| | patch | patch |
| Amount of hydrogen | 0.0506-0.1012 mg | 0.4554-0.5060 mg |
| peroxide released | | |
| Percent reduction of | 1.19-2.38% | 11.38-12.65% |
| hydrogen peroxide | | |

The results in Table 4 shows that the amount of hydrogen peroxide released from the inventive jelly-type tooth-bleaching patch into saliva after attachment to teeth is about 10% level of that from the dry-type patch.

3. Concentration of Hydrogen Peroxide in Saliva

The concentration of hydrogen peroxide in saliva was calculated from the results of Table 3, and the results are given in Table 5 below. This calculation is based on the assumption that the amount of saliva collected after the lapse of each time interval is 1 g.

TABLE 5

| | Concentration of |
|---|---|
| | hydrogen peroxide |
| Jelly-type tooth-bleaching patch | 8.43-16.87 ppm |
| Dry-type tooth-bleaching patch | 75.9-84.33 ppm |

The results in Table 5 above indicates that the concentration of hydrogen peroxide in saliva for the dry-type patch is about 10 times higher than that for the jelly-type patch. Such a high concentration of hydrogen peroxide will irritate the mouth upon the use of the dry-type patch.

When putting the above experimental results together, it can be found that the inventive jelly-type tooth-bleaching patch upon attachment on teeth releases hydrogen peroxide into saliva at 10% level of the dry-type tooth-bleaching patch, suggesting that a sufficient amount of hydrogen peroxide to bleach teeth comes in contact with the teeth. Also, this suggests that the dry-type patch irritates the mouth due to a high concentration of hydrogen peroxide in saliva.

As described above, the jelly-type tooth-bleaching patch according to the present invention has a similar adhesiveness to a poultice product for attachment to the skin, so that the sticking of drugs to hand can be minimized. Also, the adhesive in the jelly-type patch is applied on the thin polyethylene film, so that the jelly-type patch can cover all teeth and can also be attached to the front of the teeth. Furthermore, the time taken for hydrogen peroxide in the jelly-type patch to dissolve in water is at least three times longer than that of a gel-type strip, and at least two times longer than that of a dry-type patch, and the contact time of hydrogen peroxide with teeth is so much longer, suggesting that it shows a maximized tooth-bleaching effect even with a small amount of the tooth-bleaching component. In other words, the content of hydrogen peroxide in the jelly-type tooth-bleaching patch is reduced slowly while the adhesive is dissolved with the absorption of saliva, so that a sufficient concentration of hydrogen peroxide to bleach teeth will come in contact with the teeth for a sufficient time. Thus, the jelly-type tooth-bleaching patch has an excellent tooth-bleaching effect as compared to that of any existing tooth-bleaching products. Moreover, the present invention provides the jelly-type tooth-bleaching patches with the distinction between a medical product and a non-medical product according to the content of hydrogen peroxide in the adhesive and the kind of the coating-forming agent, to consider the user's safety.

What is claimed is:

1. A jelly-type tooth-bleaching patch comprising: a film, an adhesive applied on the film, and a moisture-proof wrapping paper adhered on the adhesive.

2. The jelly-type tooth-bleaching patch of claim 1, wherein the adhesive comprises peroxide, a peroxide stabilizer, an inhibitor of tartar formation, a coating-forming agent, a coating-forming aid, a wetting agent, and a plasticizer.

3. The jelly-type tooth-bleaching patch of claim 1, wherein the adhesive comprises a coating-forming peroxide, a peroxide stabilizer, an inhibitor of tartar formation, a coating-forming agent, a coating-forming aid, a wetting agent, and a plasticizer.

4. The jelly-type tooth-bleaching patch of claim 2, wherein the peroxide is at least one compound selected from the group consisting of hydrogen peroxide, carbamide peroxide, calcium peroxide, sodium percarbonate, sodium perborate, tetra sodium pyrophosphate peroxide, hydrogen peroxide 2-pyrolidinone 1-ethenyl-homopolymer, PVP-VA-hydrogen Peroxide®, Gafquat HS-100-hydrogen peroxide®, PVP-AA-hydrogen peroxide®, and Ganex-hydrogen peroxide®.

5. The jelly-type tooth-bleaching patch of claim 2, wherein the peroxide stabilizer is at least one compound selected from the group consisting of sodium stannate, alkylarylsulfonate, alkylarylsulfonate salt, alkyldiphenyloxide disulfonate, Span 20®, Span 40®, Span 60®, Span 85®, Tween®, and polyoxyethylene glycol hydroxymethylcellulose.

6. The jelly-type tooth-bleaching patch of claim 2, wherein the inhibitor of tartar formation is sodium salt, calcium salt, or a mixture thereof.

7. The jelly-type tooth-bleaching patch of claim 6, wherein the sodium salt is at least one compound selected from the group consisting of tetrasodium pyrophosphate (TSPP), sodium hexametaphosphate (SHMP), sodium acid pyrophosphate (SAPP), sodium pyrophosphate (TKPP), acidic sodium meta-polyphosphate, and acidic sodium polyphosphate.

8. The jelly-type tooth-bleaching patch of claim 6, wherein the calcium salt is at least one compound selected from the group consisting of potassium benzoate, potassium citrate, potassium fluoride, potassium bicarbonate, potassium carbonate, potassium chloride, potassium lactate, potassium pyrophosphate, and potassium phosphate.

9. The jelly-type tooth-bleaching patch of claim 2, wherein the coating-forming agent is at least one compound selected from the group consisting of polyvinyl pyrrolidone, polyvinyl alcohol, sodium alginate, hydrogen peroxide 2-pyrolidinone 1-ethenyl-homopolymer, Gafquat HS-100-hydrogen peroxide®, PVP-AA-hydrogen peroxide®, and Ganex-hydrogen peroxide®.

10. The jelly-type tooth-bleaching patch of claim 2, wherein the coating-forming aid is at least one compound selected from the group consisting of methyl cellulose, dextrin, urea, hydroxy propyl starch, gelled starch, carboxymethyl cellulose, carboxypropyl cellulose, poloxamer, carrageenan, vee gum, karaya gum, xanthan gum, guar gum, hydroxypropylmethyl cellulose, gum arabic, gum fra, hydroxyethyl cellulose, hydroxypropyl cellulose, hydroxymethyl cellulose, phthalate acetate cellulose, ethyl cellulose, phthalate hydroxypropylmethyl cellulose, calcium carboxymethyl cellulose, and sodium carboxymethyl cellulose.

11. The jelly-type tooth-bleaching patch of claim 2, wherein the wetting agent is at least one compound selected from the group consisting of polyethylene glycol, glycerin, polypropylene glycol, polybutylene glycol (1,3-butandiol), bentonite, and sorbitol.

12. The jelly-type tooth-bleaching patch of claim 2, wherein the plasticizer is at least one compound selected from the group consisting of caster oil, hydrogenated caster oil, and silicone oil.

13. The jelly-type tooth-bleaching patch of claim 3, wherein the coating-forming peroxide is at least one compound selected from the group consisting of hydrogen peroxide 2-pyrolidinone 1-ethenyl-homopolymer, PVP-VA-hydrogen peroxide®, Gafquat HS-100-hydrogen peroxide®, PVP-AA-hydrogen peroxide®, and Ganex-hydrogen peroxide®.

14. The jelly-type tooth-bleaching patch of any one of claims 2 to 13, wherein the adhesive additionally contains a sweetening agent.

15. The jelly-type tooth-bleaching patch of claim 2, wherein the adhesive comprises 0.5-10 wt % of the peroxide, 0.5-5 wt % of the peroxide stabilizer, 0.1-1.5 wt % of the inhibitor of tartar formation, 6.1-15 wt % of the coating-forming agent, 1-10 wt % of the coating-forming aid, 10-70 wt % of the wetting agent, 0.1-3 wt % of the plasticizer, and 0.01-1 wt % of the sweetener.

16. The jelly-type tooth-bleaching patch of claim 15, wherein the adhesive comprises hydrogen peroxide at the amount of 0.5-3 wt % relative to the total weight of the adhesive.

17. The jelly-type tooth-bleaching patch of claim 15, wherein the adhesive comprises hydrogen peroxide at the amount of 3-10 wt % relative to the total weight of the adhesive.

18. The jelly-type tooth-bleaching patch of claim 2, wherein the adhesive additionally comprises a crosslinking agent and a crosslinking aid.

19. The jelly-type tooth-bleaching patch of claim 18, wherein the coating-forming agent contains at least one compound selected from the group consisting of polyacrylic acid, sodium polyacrylate, polyacrylic acid/sodium polyacrylate copolymer, PVP/VA copolymer, VP/acrylates/lauryl methacrylate copolymer, polymethyl vinyl ether/maleic anhydride, vinyl acetate/vinyl neodecanoate/maleic half-ester, poly N-vinyl formamide, vinyl acetate/crotonic acid/vinyl neodecanoate copoylmer, polyvinyl acetate, polymethyl methacrylate, methacrylic acid copolymer, aminoalkyl methacrylate copolymer, cellulose acetate phthalate, shellac, vinyl acetate/crotonic acid/vinyl propionate copolymer, and acryloyl ethyl betaine/methacrylate copolymer.

20. The jelly-type tooth-bleaching patch of claim 18, wherein the crosslinking agent is at least one compound selected from the group consisting of salicylic acid, gluconic acid, silicic acid, malic acid, tartaric acid, glycolic acid, and lactic acid.

21. The jelly-type tooth-bleaching patch of claim 18, wherein the crosslinking aid is at least one compound selected from the group consisting of kaoline, hydroxy aluminum aminoacetate, dried aluminum hydroxide powder, aluminum silicate, aluminum-magnesium hydroxide, synthetic hydrotalcite, cum lite®, and aluminum hydroxide gel.

22. The jelly-type tooth-bleaching patch of claim 18, wherein the adhesive comprises 0.1-10 wt % of the peroxide, 0.5-5 wt % of the peroxide stabilizer, 0.1-5 wt % of the inhibitor of tartar formation, 0.1-10 wt % of the coating-forming agent, 0.1-10 wt % of the coating-forming aid, 10-70 wt % of the wetting agent, 0.1-3 wt % of the plasticizer, 0.1-2 wt % of the sweetener, 0.1-1 wt % of the crosslinking agent, and 0.01-0.6 wt % of the crisslinking aid.
